(19) United States
(12) Patent Application Publication
(10) Pub. No.: US 2019/0240441 A1
(43) Pub. Date: Aug. 8, 2019

(54) MASK FOR ARTIFICIAL VENTILATION OF A PATIENT

(71) Applicant: CENTRE HOSPITALIER UNIVERSITAIRE DE BORDEAUX, TALENCE CEDEX (FR)

(72) Inventor: ERIC DUMAS DE LA ROQUE, BORDEAUX (FR)

(21) Appl. No.: 16/341,912

(22) PCT Filed: Oct. 13, 2017
(86) PCT No.: PCT/EP2017/076234

§ 371 Date: Apr. 14, 2019

(30) Foreign Application Priority Data
Oct. 14, 2016 (EP) 16306360.5

(51) Int. Cl. A61M 16/06; A61M 16/08

(52) U.S. Cl. CPC A61M 16/0688; A61M 16/0616; A61M 2240/00; A61M 2210/0618; A61M 16/0816

(57) ABSTRACT

The invention relates to a mask (100) for the mechanical ventilation of a patient, comprising:
a mask body made of flexible material, comprising a ventilation chamber (103), said mask body being provided with means (105) for connection to a respirator, and
adhesive and gas-tight attachment means (10), comprising a peripheral edge (11) secured to the mask body and intended to surround at least the patient's nostrils, in order to hold in a tight manner the ventilation chamber at least around patient's nostrils.

The invention relates to a respiratory mask for the mechanical ventilation of a patient, especially an infant. More specifically, the invention relates to a nasal or face mask, able to be held without pressure on the face of a patient, to allow the non-invasive ventilation of said patient, when said mask is connected to a respirator.

Non-invasive ventilation (NIV) is intended to increase the alveolar ventilation without using an intratracheal device. A nasal or face mask is held around the nose and/or mouth of the patient in order to deliver positive pressure into his airway through a respirator. Generally, NIV is used to optimize lung volumes and reduce breathing work of the patient.

The non-invasive side of this technique makes it particularly suitable for use in infants, newborns and especially premature newborns. Thus, NIV is generally used in infants with chronic respiratory insufficiency, respiratory distress of the newborn, acute respiratory insufficiency (severe acute asthma, acute pulmonary edema (APE), bronchiolitis), respiratory infections, ENT malformations of the Pierre Robin type, etc.

Currently, the great majority of infants and newborns treated with NIV are fitted with a rigid nasal mask held by pressure around the nose. For this, the mask is attached to a cap, the dimensions of which must allow the front part of said cap to reach the upper line of the eyebrows and the back part to fully encompass the infant's skull, with the sides covering the ears. Lateral straps (horizontal to the nose) hold the mask around the nose while encompassing it fully and allow tightness by induction of pressure on the mask. However, the adjustment of the straps is difficult, and holding the mask by pressure may induce pain and/or skin lesions. In addition these devices involve limiting the mobility of the head, the movements may otherwise cause leakage at the mask.

It is therefore known to regularly alternate (generally every two hours in the premature newborn) the use of such a nasal mask with that of a nosepiece, in order to reduce the risks of injury at the pressure points, especially at the skin in contact with the periphery of the mask. This alternation of devices is however not without inconvenience. Indeed, multiple and repeated manipulations to change the device contribute to the discomfort of the child and increase the risks of gas leakage responsible for a less effective NIV. In addition, the use of a nosepiece can cause significant risks of internal nasal lesions, especially in premature newborns.

To date, there is no face or nasal mask adapted for the non-invasive mechanical ventilation of a patient, which is compatible with a prolonged use, limits the risks of gas leakage and allows head mobility.

The aim of the invention is to at least partially solve the problem stated above by proposing a nasal or face mask, particularly adapted to NIV, whose holding on the patient's face is assured without pressure and allows a better mobility of the head. More specifically, the mask according to the invention is held around the nasal orifices (or nostrils), of the patient's nose and/or mouth by means of an adhesive surrounding at least partially the patient's nose and/or mouth. The biocompatible and gas-tight adhesive extends along the periphery of the ventilation chamber and allows holding said chamber in position on the patient's face, without risk of leakage. In addition, according to the invention, the ventilation chamber is made of flexible material, which allows improving the comfort of the patient and allows him to move the head, without the risk of moving the mask and causing gas leakage.

The invention therefore relates to a mask for the mechanical ventilation of a patient, comprising:
a mask body made of flexible material, comprising a ventilation chamber, said mask body being provided with means for connection to a respirator, and
adhesive and gas-tight attachment means, comprising an adhesive peripheral edge, secured to the mask body, and intended to surround at least the patient's nostrils, in order to hold in a tight manner the ventilation chamber at least around the patient's nostrils.

According to the invention, the wall(s) of the mask body are made of flexible material and provide an open cavity, forming a ventilation chamber. Thus, the ventilation chamber of the mask according to the invention is made of flexible material, allowing a certain deformation of said chamber. The opening of the cavity is intended to surround at least the patient's nostrils. Once the mask is applied against the patient's face, the cavity is closed by the skin of said patient. Advantageously, when using the mask on a patient, the deformability of the ventilation chamber allows it to inflate, and this inflation confirms the absence of leakage during gas inflow.

In the context of the invention, by “ventilation chamber” is meant the part of the mask in which the gases flowing into and flowing out of the patient's airway will get mixed up.

According to the invention, the dimensions of the mask body are adapted to be able to surround the nostrils of a patient, the whole nose of a patient or the nose and mouth of a patient, so as to form a nasal or face mask, as needed.

By “flexible material” is meant a material that has the ability to elastically deform in a reversible manner and without breaking. Advantageously, the flexible material used to form the mask body, and more particularly the ventilation chamber, is gas-tight and/or waterproof. Generally, any flexible material may be used, including any flexible plastic material. Of course, any other material having such properties may be used, including any biocompatible material, of natural origin or not. In one embodiment, the material used is a transparent material, through which it is possible to see the part(s) of the patient's face.

The mask body is secured, reversibly or irreversibly, to adhesive and gas-tight attachment means intended to hold the ventilation chamber around the nostrils, nose and/or mouth of a patient. Advantageously, the adhesive attachment means are also waterproof.

By “adhesive attachment means” is meant means having an adhesive surface intended to be applied to part of a patient's face, without exerting compression thereon.

The adhesive attachment means must also be gas-tight, so as to prevent any gas leakage at the junction of the mask with the patient's face. Also, the attachment means must continuously surround all the contour of the mask body and more precisely the periphery of the cavity, so as to extend along the opening of the cavity and ensure that, once attached to the patient's face, the ventilation chamber does not let any gas escape to the outside. For this, the attachment means have at least one adhesive peripheral edge, the dimensions of which follow the dimensions of the mask body part intended to be applied against the patient's face (nostrils, nose and/or mouth). Of course, the peripheral edge of the attachment means forms a central orifice sufficient to let pass, according to the embodiment, the nostrils, nose and/or mouth of the patient. According to the invention, the adhesive attachment means are directly attached to the mask body that is to say to the walls made of flexible material that form the ventilation chamber.

In one embodiment, the attachment means further have one or more adhesive tab(s) extending radially from the adhesive peripheral edge. This or these adhesive tab(s) is/are intended to extend over parts of the patient's face more or less away from the patient's nose and/or mouth, in order to increase the adhesion surface and thus enhance the good attachment of the mask on the patient's face.

In one embodiment, the attachment means comprise at least one adhesive tab intended to extend along the bridge of the patient's nose. Alternatively or complementarily, the attachment means comprise at least two adhesive tabs intended to extend on either side of the nose alae, the nose and/or the mouth of the patient. According to the invention, any adhesive material that can be applied directly to the skin of a human being can be used to form the adhesive part of the attachment means. Advantageously, the adhesive material is a biocompatible adhesive material, meeting the ISO 10993 standard. By biocompatible material, is meant a material that is suitable for use in or on biological tissues, without degrading the concerned biological tissues during or after the contact. In the context of the invention, the biocompatible material used must in particular take into account the properties of the patient's skin. In one embodiment, the adhesive surface of the adhesive attachment means is made of polyurethane, acrylic, cyanoacrylate, hydrocolloid material, etc. The person skilled in the art knows which biocompatible adhesive material to use depending on the total adhesive surface to be provided, on the fragility of the patient's skin for whom the mask is intended, on the desired time for placing the mask, etc. According to the invention, the adhesive attachment means are made of flexible material, so as to be deformable and adaptable to the contours of the patient's face, while ensuring the tightness of the link.

The mask according to the invention therefore comprises a mask body made of flexible material, and adhesive attachment means made of flexible material. Thus, the whole mask (with the possible exception of the means for connection to a respirator that may be made of rigid, semi-rigid or flexible material) is made of flexible material. The use of such a flexible material ensures a good adaptability of said mask to the patient's face and to its movements and/or limits the weight of the mask and therefore the stress applied to the face and/or limits the stresses during the movements of the gas intake pipes.

In one embodiment, the adhesive attachment means are peelable, in the sense that the adhesive surface can be detached, after having been glued to the skin of a patient, without having to wet said attachment means and without the risk of damaging/traumatizing the skin of said patient.

Advantageously, the adhesive attachment means are repositionable, in the sense that the adhesive surface can be detached and then re-glued at least twice, preferably at least three times or more, without losing its adhesive power.

The adhesive and gas-tight attachment means according to the invention make it possible to hold the mask on the patient's face, while limiting the risks of gas leakage at the periphery of the ventilation chamber. No pressure is exerted on the patient's face on which such a mask is attached, thereby allowing a prolonged time for placing the mask, in particular between 12 to 24 hours, compared to ventilation masks held by pressure.

Advantageously, the adhesive surface of the adhesive attachment means is covered with a peelable protective film, intended to be removed at the time of use of the mask.

In one embodiment, the mask body is removably attached to the attachment means. In other words, the attachment means can be separated from the mask body, without damaging said body and/or the attachment means. This may for example be useful, when it is necessary, to remove the mask body from the patient's face, in order to perform a procedure and/or conduct special care, before putting the mask back in place, or changing it.

According to the invention, the ventilation mask is intended to be used with a respirator, linked to said mask by connection means, most often consisting of at least one gas inlet and/or outlet pipe (air plus oxygen if necessary). In one embodiment, the connection means comprise a single pipe forming a gas intake and discharge pipe. Advantageously, the mask body comprises connection means for directly bringing the gases, coming from the respirator, into the ventilation chamber.

In one embodiment, the connection means are front connection means, that is to say they allow the pipe(s) to be held upwards (towards the forehead) relative to the patient's face on which the mask is intended to be positioned.

In another embodiment, the connection means are lateral connection means, that is to say they allow the pipe(s) to be held along one or both patient's cheek(s) on which mask is intended to be positioned.

In another embodiment, the connection means are lower connection means, that is to say they make it possible to hold the pipe(s) downward (towards the chin) relative to the patient's face on which the mask is intended to be positioned.

Alternatively or complementarily, the connection means are connection means having an interface making them movable at 180 degrees.

In a particular embodiment of the mask according to the invention, the mask body is equipped with stiffening means. This may in particular be useful, according to the dimensions of the mask body, to prevent the ventilation chamber, whose walls are made of flexible material, from twisting.

For example, the mask body may comprise a double wall made of flexible material spaced from each other to form a gap in which air can circulate before entering the ventilation chamber. More specifically, the air from the respirator is blown between the two walls of the mask body, thereby stiffening the assembly. One or more hole(s) formed in the inner wall, i.e. the wall bordering the ventilation chamber, then allow(s) air to enter the ventilation chamber.

Alternatively or complementarily, it is possible to surround the mask body with stiffening rings, such as for example rings made of rigid plastic.

According to the invention, it is possible to provide on the mask body at least one passage for a probe. Advantageously, said passage is able to be hermetically sealed and gas-tight in the absence of a probe.

The mask according to the invention is particularly intended for an infant or a newborn, especially a premature newborn. Of course, such a mask can also be used on a child, a teenager or an adult. In particular, the mask according to the invention can be used on a patient undergoing treatment against sleep apnea and requiring the use of a NIV.

In general, the person skilled in the art knows how to adapt the dimensions of the mask for mechanical ventilation depending on the patient for whom it is intended.

The invention also relates to a system for the mechanical ventilation of a patient, comprising at least one mask as described above and a respirator able to be connected to said mask.

The invention will be better understood upon reading the following description and upon examining the figures which accompany it. These are presented for information purposes and are in no way limiting the invention.

The figures represent:

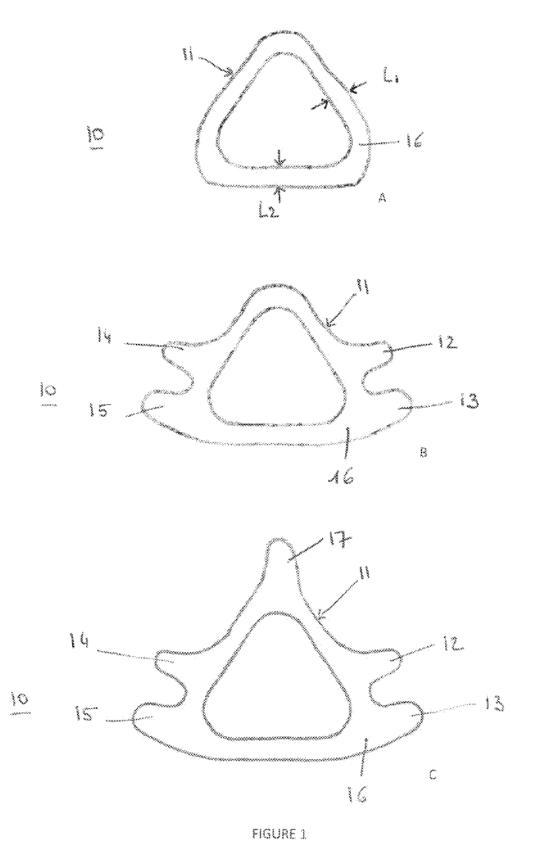

FIGS. 1A to 1C: Schematic representations, viewed from the front, of various embodiments of the adhesive and gas-tight mask attachment means for mechanical ventilation according to the invention;

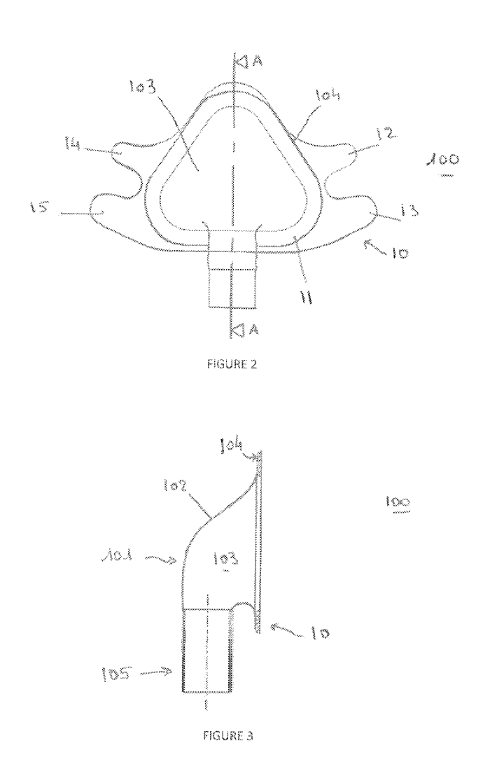

FIG. 2: Schematic representation, viewed from the front on the inner side (that is to say on the side intended to be applied against the face of a patient), of a mask for mechanical ventilation according to an exemplary embodiment of the invention;

FIG. 3: Schematic representation, in longitudinal section along the axis AA, of the mask for mechanical ventilation of FIG. 2;

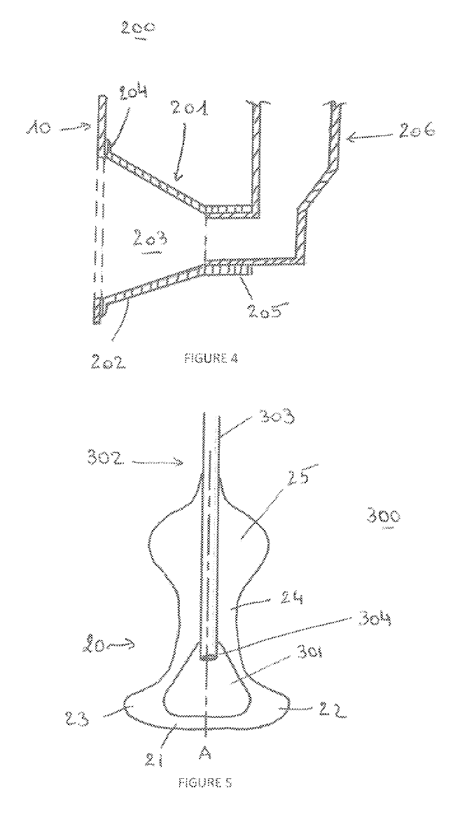

FIG. 4: Schematic representation, in longitudinal section, of a mask for mechanical ventilation according to a second exemplary embodiment of the invention;

FIG. 5: Schematic representation, viewed from the front, of a mask for mechanical ventilation according to a third embodiment of the invention.

The mask for mechanical ventilation according to the invention comprises a mask body made of flexible material, providing a ventilation chamber, and adhesive and gas-tight attachment means, comprising a peripheral edge secured to the mask body.

FIGS. 2, 3, 4 and 5 illustrate exemplary embodiments of the mask according to the invention. FIGS. 1A-1C illustrate various exemplary embodiments of adhesive means that can be used. It is understood that these adhesive means can be adapted to any shape of mask body made of flexible material, and in particular to the shapes illustrated in FIGS. 2-5.

In the embodiment represented in FIGS. 2 and 3, the ventilation mask 100 comprises a mask body 101 whose walls 102, made of flexible material, form an open cavity forming a ventilation chamber 103. The ventilation chamber 103 has dimensions that are sufficient to surround at least the nostrils of a patient. In the embodiment described here, the ventilation chamber 103 is able to surround the nose of a patient.

An outer periphery 104 of the mask body 101, bordering the ventilation chamber 103, is secured to adhesive and gas-tight attachment means 10.

In general, the attachment means have an adhesive surface intended to come into contact with the patient's face to adhere thereto and thus hold the ventilation chamber in position around the patient's nostrils, nose and/or mouth, depending on the shape of the mask. The attachment means will be described in more detail below using FIGS. 1A-1C.

In the example shown in FIGS. 2 and 3, the attachment means 10 (corresponding to the attachment means represented in FIG. 1B) comprise a peripheral edge 11 that follows the contour of the ventilation chamber 103, as well as lateral tabs 12, 13, 14, 15 extending radially outwards from the peripheral edge 11.

The mask body 101 is also provided with connection means 105 to a respirator (not shown). The connection means 105 extend vertically downwards from the mask body 101, so that said connection means 105 and the pipes that will be connected thereto extend along the patient's chin on which the mask 100 will be placed.

In the embodiment represented in FIG. 4, the mask 200 similarly has a mask body 201 and adhesive and gas-tight attachment means. The wall 202 of the mask body 201 forms a ventilation chamber 203 intended to receive the patient's nostrils, nose and/or mouth. The outer periphery 204 of the mask body 201 is secured to the peripheral edge of the attachment means 10, so that the ventilation chamber can be held by gluing around the patient's nose and/or mouth.

In this embodiment, the mask body 201 is provided with frontal connection means 205 to a respirator. More specifically, the connection means 205 vertically protrude from the apex of the mask body, so that the pipes 206 connected to said connection means 205 extend upwards along the patient's forehead on which the mask 200 will be placed.

In the embodiment represented in FIG. 5, the ventilation mask 300 comprises an open cavity forming a ventilation chamber 301. The ventilation chamber 301 has dimensions that are sufficient to surround the nose of a patient. The attachment means 20 comprise a peripheral edge 21, which follows the contour of the ventilation chamber 301, two lateral tabs 22, 23, extending radially outwards from the peripheral edge 21 and intended to extend on either side of the patient's nostrils, and a vertical tab 24, intended to extend along the bridge of the nose and on part of the patient's forehead. The mask 300 is provided with front connection means 302. More specifically, a pipe 303 is held by the vertical bridge, so that an end 304 of said pipe 303 opens into the ventilation chamber 301. Thus, the pipe 303 will extend from the ventilation chamber 301, along the bridge of the patient's nose and over a height of the forehead of said patient, towards an air inlet or a respirator. In order to ensure that the pipe 303 is properly held on the patient's face, the vertical tab 24 advantageously has a lateral flare 25 intended to increase the surface of adhesion on the patient's forehead.

In this embodiment, the force and movement stresses due to the presence of the connection means are away from the ventilation chamber, so the risks of leakage and/or mask detachment are reduced.

Examples of adhesive and gas-tight attachment means for use in the mask for mechanical ventilation according to the invention will now be described in more detail with reference to FIGS. 1A, 1B and 1C. The same numerical references are used to designate the elements in common to these three embodiments.

The adhesive attachment means 10 according to the invention are intended, on the one hand, to be attached to the mask body and, on the other hand, to adhere by gluing to the patient's face, in order to hold the mask body in position on the face of said patient. Any means for attaching the peripheral edge of the means for attachment to the mask body may be envisaged, and in particular a reversible means, such as by clipping, or irreversible means such as by gluing. In all cases, the link between the peripheral edge of the attachment means and the mask body must be gas-tight.

According to the invention, the attachment means comprise a peripheral edge, a general contour of which follows the general contour of the wall of the mask body bordering the cavity forming the ventilation chamber. The peripheral edge of the attachment means forms an orifice or recess intended to surround, according to the mask, the patient's nostrils, nose and/or mouth. A width of the peripheral edge may vary according to the attachment means, but also within the same attachment means. For example, in the case of a nasal mask, it is possible to provide a first width L1 at the part of the peripheral edge intended to extend on either side of the patient's nose alae and a second width L2 at the part of the peripheral edge intended to extend along the upper lip (FIG. 1A). It may then be interesting to have a first width L1 greater than the second width L2, especially in the case where the patient has a weak skin surface between the base of the nose and the upper lip, as it is the case in infants and newborns.

In the example represented in FIG. 1A, the attachment means 10 consist only of the peripheral edge 11. The adhesive surface 16 is therefore limited to the area surrounding precisely the part of the face (nostrils, nose and/or mouth) against which the ventilation mask is applied.

In the example represented in FIG. 1B, lateral tabs 12, 13, 14, 15 extend radially from the peripheral edge 11, said tabs 12, 13, 14, 15, also adhesive, being intended to stick on the patient's cheeks. The surface of adhesion with the patient's skin is thus increased compared to that of Example 1A, improving the good attachment of the mask on the patient's face. Of course, it is possible to provide more, on the contrary less, lateral tabs. It is also possible to act on the dimensions and shape of these tabs.

In the example represented in FIG. 1C, the attachment means 10 comprise, in addition to the lateral tabs 12, 13, 14, 15, a vertical tab 17 intended to extend along the bridge of the patient's nose, and possibly a total or partial height of the patient's forehead. By height is meant the dimension extending parallel to the longitudinal axis (A) of the mask. It is also possible to provide attachment means comprising only the peripheral edge and the vertical tab.

The person skilled in the art will be able to adapt the dimensions of the peripheral edge as well as the number, shape, position and dimensions of the tabs according to the (face or nasal) mask but also depending on the patient (infant, premature newborn, child, teenager, adult, etc.).

1-11. (canceled)

12. A mask for the mechanical ventilation of a patient, comprising:
a mask body made of flexible material, comprising a ventilation chamber, said mask body being provided with means for connection to a respirator, and
adhesive and gas-tight attachment means, comprising a peripheral edge secured to the mask body and intended to surround at least the patient's nostrils, in order to hold in a tight manner the ventilation chamber at least around the patient's nostrils.

13. The mask according to claim 12, wherein the dimensions of the peripheral edge are such that said peripheral edge is able to surround the patient's nostrils and nose, in order to hold in a tight manner the ventilation chamber around the patient's nose.

14. The mask according to claim 12, wherein the dimensions of the peripheral edge are such that said peripheral edge is able to surround the patient's nostrils, nose and/or mouth, in order to hold in a tight manner the ventilation chamber around the patient's nose and mouth.

15. The mask according to claim 12, wherein the mask body is removably attached to the attachment means.

16. The mask according to claim 12, wherein the attachment means further comprises at least one adhesive tab extending radially from the peripheral edge, said tab being intended to extend over the nose bridge and/or the nose alae and/or the face of the patient, so as to increase the adhesive surface of the attachment means.

17. The mask according to claim 12, wherein the attachment means are peelable.

18. The mask according to claim 12, wherein the attachment means are repositionable.

19. The mask according to claim 12, wherein the means for connection to a respirator are front, lateral or bottom connection means.

20. The mask according to claim 12, wherein the means for connection to a respirator have an interface making them movable at 180 degrees relative to the respirator.

21. The mask according to claim 12, wherein the mask body is provided with stiffening means.

22. The mask according to claim 21, wherein the mask body comprises a double wall made of flexible material forming a gap in which air can circulate before entering the ventilation chamber.

23. The mask according to claim 21, wherein the mask body is surrounded by stiffening rings.

24. The mask according to claim 12, wherein the mask body further comprises at least one passage for a probe.

25. The mask according to claim 12, said mask being intended for a newborn, especially a premature newborn.

26. A system for the mechanical ventilation of a patient, comprising at least one mask according to claim 12, and a respirator able to be connected to said mask.
